(19) United States
(12) Patent Application Publication
(10) Pub. No.: US 2019/0321593 A1
(43) Pub. Date: Oct. 24, 2019

(54) SYSTEMS AND METHODS FOR A TELESCOPING DRAINAGE CATHETER AND ASSOCIATED LOCKING MECHANISMS

(71) Applicant: Daniel Crawford, Scottsdale, AZ (US)

(72) Inventor: Daniel Crawford, Scottsdale, AZ (US)

(21) Appl. No.: 16/388,782

(22) Filed: Apr. 18, 2019

Related U.S. Application Data
(60) Provisional application No. 62/660,334, filed on Apr. 20, 2018.

(51) Int. Cl. A61M 25/00; A61M 25/04

(52) U.S. Cl. CPC A61M 25/0074; A61M 25/003; A61M 25/0068; A61M 2025/0039; A61M 25/0097; A61M 25/0009; A61M 25/04

(57) ABSTRACT

Embodiments of a system and method for a telescoping drainage catheter and associated locking mechanism designed to reduce catheter dislodgement or withdrawal by lengthening when longitudinal tension is applied to the drainage catheter. The drainage catheter includes a telescoping element and flexible connection between the telescoping components for placing the drainage catheter between extended and non-extended states.

### CROSS REFERENCE TO RELATED APPLICATIONS

This is a non-provisional application that claims benefit to U.S. provisional patent application Ser. No. 62/660,334 filed on Apr. 20, 2018, which is herein incorporated by reference in its entirety.

### FIELD

The present disclosure generally relates to catheters, and more particularly to drainage catheters and associated locking mechanisms.

### BACKGROUND

Implantable catheter stents or drainage tubes shunt fluid from one body cavity to another or to the outside environment. Several examples include nephrostomy catheters, nephroureteral catheters, abscess drainage catheters, and cholecystostomy catheters. Upon implantation, these types of catheters are effective at draining fluids; however, a common complication is movement of the catheter from its desired location in a body cavity, which can result in catheter dislodgement or withdrawal from its intended location. Catheter dislodgement or withdrawal often requires a subsequent procedure to replace or remove the catheter.

Various attempts have been made to decrease catheter dislodgement and withdrawal. For example, a catheter with a pigtail loop at its distal end is often used. After the catheter is inserted into the body cavity, the pigtail loop is formed by pulling on a proximal end of a fiber. This fiber extends through and then out of the catheter, attaching near the distal end of the catheter. In addition, this fiber can then be secured to the proximal end of the catheter by various means, which holds the fiber in place and retains the loop shape at the distal end of the catheter. The pigtail loop requires a higher force to be pulled out of the body cavity and can help reduce catheter dislodgement or withdrawal. Other types of retention mechanisms include other catheter shape memories or balloons near the distal tip.

Another method that attempts to reduce movement of the catheter from its desired location is to more securely attach the proximal end of the catheter to the skin near the catheter exit site. Typically, to secure a catheter to a patient, a suture is tied through the skin and then around the catheter near the skin exit site. However, if tension is applied to the catheter, the suture may sometimes slide along the catheter and result in catheter dislodgement or withdrawal. Various anchoring mechanisms have been described that secure a catheter to a patient by more firmly grasping the catheter with clamps or ties that increase the friction between the catheter and the anchoring mechanism. These devices must then be secured to the skin through adhesives or sutures.

As such, catheter dislodgement or withdrawal still frequently occurs despite these attempts to address the issue. Unfortunately, there is currently no satisfactory method or system to prevent unwanted catheter dislodgement or withdrawal.

It is with these observations in mind, among others, that various aspects of the present disclosure were conceived and developed.

### BRIEF DESCRIPTION OF THE DRAWINGS

FIG. 1A is a side view of one embodiment of a drainage catheter in a non-extended state.

FIG. 1B is a side view of the drainage catheter of FIG. 1A in an extended state.

FIG. 2A is an enlarged cross-sectional view showing a telescoping element of the drainage catheter in the non-extended state.

FIG. 2B is an enlarged cross sectional view showing the telescoping element of FIG. 2A in the extended state.

FIGS. 3-10 are enlarged cross-sectional views of multiple embodiments of telescoping elements for the drainage catheter in non-extended states.

FIG. 11A is an enlarged cross-sectional view of the telescoping element in the non-extended state.

FIG. 11B is an enlarged cross-sectional view of the telescoping element of FIG. 11A in the extended state.

FIG. 12 is an enlarged cross-sectional view of an embodiment of a mechanism that prevents catheter component separation of the drainage catheter.

FIG. 13 is an enlarged cross-sectional view of another embodiment of a mechanism that prevents catheter component separation of the drainage catheter.

FIG. 14 is an enlarged perspective view of an embodiment of the drainage catheter having a mechanism that minimizes rotation between the telescoping elements.

FIG. 15A is an enlarged perspective view of a disassembled drainage catheter having a mechanism that prevents catheter component separation and minimizes rotation between the telescoping elements.

FIG. 15B is an enlarged cross-sectional view of the telescoping drainage catheter shown in FIG. 15A in an assembled state.

FIG. 16A is a side view of one embodiment of an embodiment of a drainage catheter having an automated locking mechanism with the drainage catheter in the non-extended state prior to formation of a pigtail loop.

FIG. 16B is the catheter shown in FIG. 16A with the drainage catheter in the non-extended state after the pigtail loop has been formed.

FIG. 16C is the drainage catheter shown in FIG. 16A with the drainage catheter in the extended state after the pigtail loop has been formed.

FIGS. 17A, 17B, and 17C are enlarged cross-sectional views of an auto-locking mechanism for the drainage catheter.

FIG. 18A is a simplified illustration of one embodiment of a drainage catheter in the non-extended state disposed within a cavity of a patient.

FIG. 18B is a simplified illustration of the drainage catheter depicted in FIG. 18A in the extended state after longitudinal tension has been placed on the drainage catheter and the drainage catheter has been lengthened without dislodgement or withdrawal.

Corresponding reference characters indicate corresponding elements among the view of the drawings. The headings used in the figures do not limit the scope of the claims.

### DETAILED DESCRIPTION

One aspect of the present disclosure relates to a drainage catheter having a telescoping capability that reduces catheter dislodgement or withdrawal by lengthening the body of the drainage catheter when longitudinal tension is applied to the drainage catheter. In some embodiments, the drainage catheter includes a telescoping element and a flexible connection between the distal and proximal catheter components for placing the drainage catheter between extended and non-extended states.

In some embodiments, the drainage catheter includes a telescoping section and a flexible connection positioned between the proximal and distal catheter components having a fiber that forms and locks into place a loop shape along the free end portion of the distal catheter component. In some embodiments, the fiber attaches near the proximal hub of the drainage catheter and has sufficient extra length for the drainage catheter to lengthen, but applies tension on the loop shape when the drainage catheter lengthens to a sufficient degree.

For example, embodiments are described herein in connection with drainage catheters. However, embodiments within the scope of this disclosure can be applied toward any type of catheter, tube, or mechanism of similar structure and/or function. Furthermore, embodiments within the scope of this disclosure can be applied to other applications, including but not limited to chest tubes, plastic stents, nephroureteral stents, biliary stents, intravenous catheters, arterial catheters, urinary catheters, epidural catheters, and enteral tubes.

A drainage catheter, or any portion thereof as disclosed herein can be made of any number of materials including silicone, latex, polyurethanes, polyvinyl chlorides, polyethylenes, polysiloxanes, polycarbonates, nylons, PTFE, ePTFE, PEEK, stainless steel, cobalt chromium, nitinol, or any other biocompatible material, including combinations of the foregoing. The construction of a drainage catheter, or any portion thereof, can be a single extrusion, laminated, a composite, include discrete circular bands, include a helical wrap, a braid, or any other combination or construction that achieves desired functional goals. Various inner and/or outer liners may be used at select positions along the drainage catheter to affect material properties and/or friction between catheter components. Additionally, a drainage catheter, or any portion thereof, can have hydrophilic or hydrophobic properties.

In describing various embodiments, the term “distal” is used to denote the end of a device nearest to the treatment region within a patient's body, while the term “proximal” is used to denote the end of a device nearest to the user or operator of the device.

In addition, the term “telescoping element” as used herein in the context of drainage catheters includes a catheter component that can move longitudinally inside another catheter component. By definition, as used herein an inner component can move inside an outer component. A component can be, for example, a tube or cover. In some embodiments, a tube can have a variety of cross-sectional shapes including but not limited to circular, oval, triangular, square, polygonal, uniform, or random shapes. In some embodiments, radiopaque markers can be integrated to the inner component and/or outer component to aid the operator in identifying the relative locations of the telescoping components.

The term “cover” as used herein refers to a material extending from one catheter component to another catheter component. In some embodiments, the cover stiffness may be flexible, semi-rigid, or any stiffness in between. As used herein, the term “cover” may be synonymous with flexible catheter component.

As used herein, the term “couple” means to join, connect, attach, adhere, affix, or bond, whether directly or indirectly, and whether permanently or temporarily.

As illustrated herein, certain components that are not visible are represented as dashed lines.

Telescoping Drainage Catheter

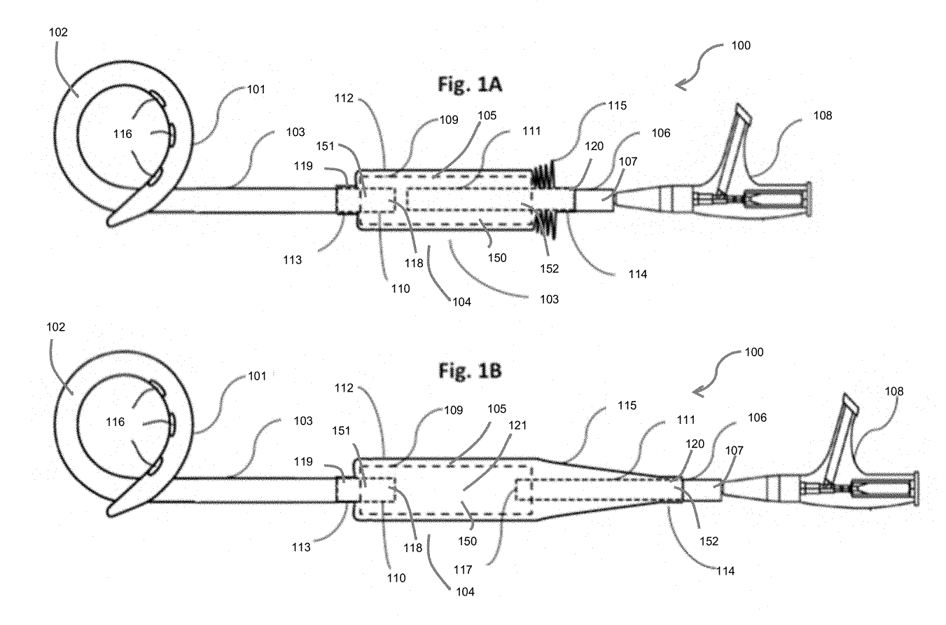

Referring to the drawings, various embodiments of a drainage catheter 100 are illustrated and generally indicated at 100 in FIGS. 1-18. The drainage catheter 100 as shown in FIGS. 1A-1B comprises a telescoping element 104 that defines an outer telescoping component 109 coupled to the proximal end 110 of a distal catheter component 103 and to the distal end 111 of a proximal catheter component 106, and a cover 112 coupled to the distal catheter component 103. The outer telescoping component 109 further defines an interior portion 150, an inner lumen 105, a distal opening 151, and a proximal opening 152. The distal end 111 of the proximal catheter component 106 is slidably disposed within the outer telescoping element 109. The cover 112 further defines an excess cover portion 115, a distal end 119 attached to a distal catheter component 103, and a proximal end 120. The cover 112 may extend over the outer telescoping element 109 and can be coupled to the proximal end of the proximal catheter component 106 at site 114. In some embodiments, the cover 112 may further include an excess cover length portion 115.

The proximal end 120 of the proximal catheter component 106 is coupled to the cover 112 of the outer telescoping component 109 at site 114. The proximal catheter component 106 comprises an inner lumen 107 in fluid connection with a proximal hub 108. The distal end 111 of the proximal catheter component 106 is disposed within the telescoping component 109. In some embodiments, a shorter length of the distal end 111 of the proximal catheter component 106 is disposed within the outer telescoping element 109. Material that might otherwise egress between the outer telescoping component 109 and the distal end 111 of the proximal catheter component 106 is contained by the presence of the cover 112, thereby preventing material communication outside of the telescoping element 104.

In some embodiments, the proximal end 118 of the distal catheter component 103 is engaged to the outer telescoping component at site 110. The distal catheter component 103 comprises an inner lumen 102 which is in fluid flow communication with the inner lumen 105 of the outer telescoping component 109. The distal catheter component 103 also is configurable to form a distal pigtail loop 101 which defines a plurality of drain holes 116 as shown in FIG. 1A in communication with the inner lumen 102 of the distal catheter component 103.

The excess cover length portion 115 can be in an extended or non-extended state. When the excess cover length portion 115 is in the non-extended state, it can assume a compressed non-extended state wherein the excess cover length portion 115 has retracted towards the telescoping element 104 and away from the proximal catheter component 106 as illustrated in FIG. 1A. As shown in FIG. 1B, the overall length of the drainage catheter 100 has increased when the excess cover length portion 115 is in the extended state. Further, the space 121 between the proximal end 118 of the distal catheter component 103 and the distal end 111 of the proximal catheter component 106 has increased. The excess cover length portion 115 is straightened and no longer in a compressed non-extended state when in the extended state. In the extended state, the distal edge of the proximal catheter component 106 stays disposed within the outer telescoping component 109 because the cover 112 has reached its maximum length when attached along sites 113 and 114.

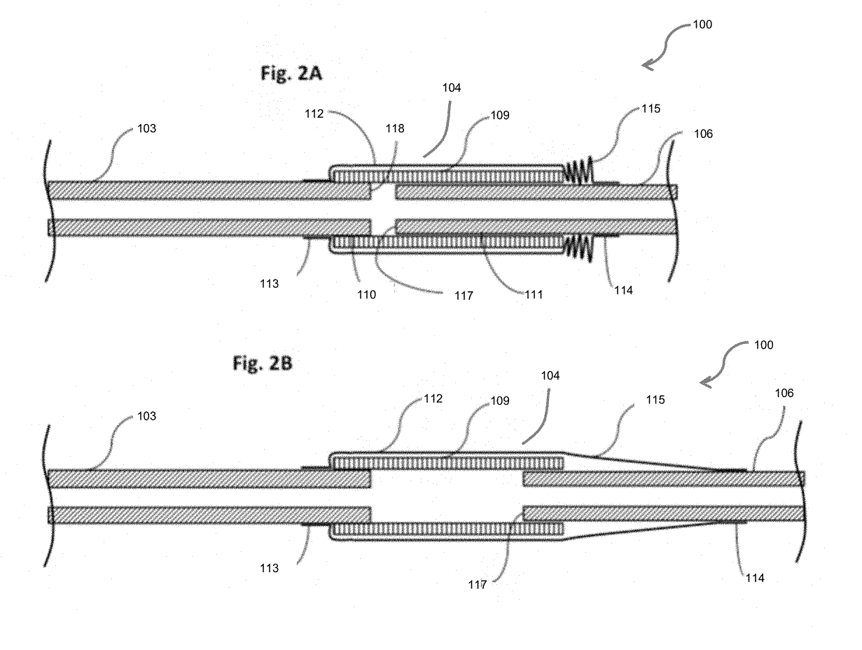

FIG. 2A shows the telescoping element 104 of the drainage catheter 100 of FIG. 1A in the non-extended state. In the extended state, the outer telescoping component 109 is coupled to the proximal end of the distal catheter component 103 at site 110, while the cover 112 is coupled to the distal catheter component 103 at site 113. The cover 112 includes an excess cover length portion 115 that is in the “scrunched up” compressed state while the drainage catheter 100 is in the non-extended state. The distal end 117 of the proximal catheter component 106 is shown separated from the proximal end 118 of distal catheter component 103. In the non-extended state of the drainage catheter 100, the proximal and distal ends 117 and 118 may contact each to improve pushability of the drainage catheter 100 during introduction. In some embodiments, these edges 117 and 118 may have different shapes, tapers, and angles in order to facilitate pushability while minimizing frictional interference between the proximal and distal catheter components 106 and 103, respectively, when they are separated longitudinally.

FIG. 2B illustrates the telescoping element of FIG. 2A in the extended state. The distal catheter component 103 and the proximal catheter component 106 are shown longitudinally separated. The excess cover length portion 115 straightens out during extension of the drainage catheter 100 to the extended state because the cover 112 is coupled to the distal catheter component at 113 and the proximal catheter component at site 114. In this embodiment, the distal end 117 of the proximal catheter component 106 does not extend past the end of the outer telescoping component 109 due to the tension in the cover 112.

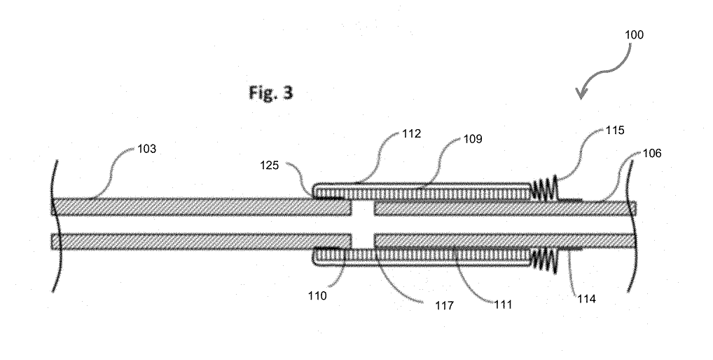

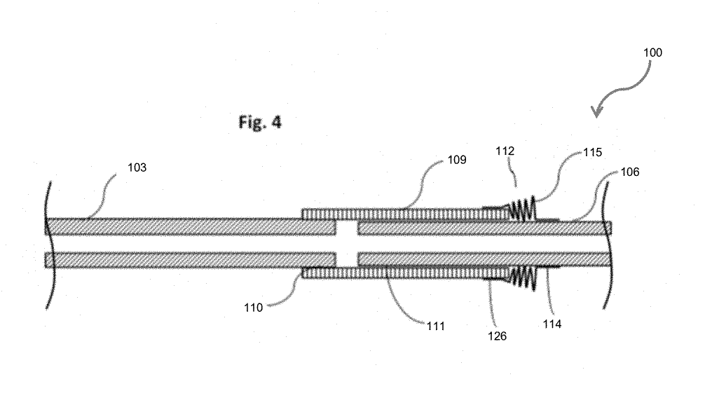

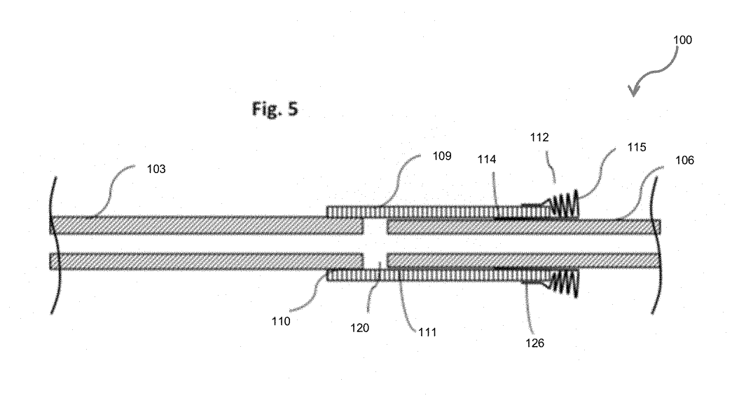

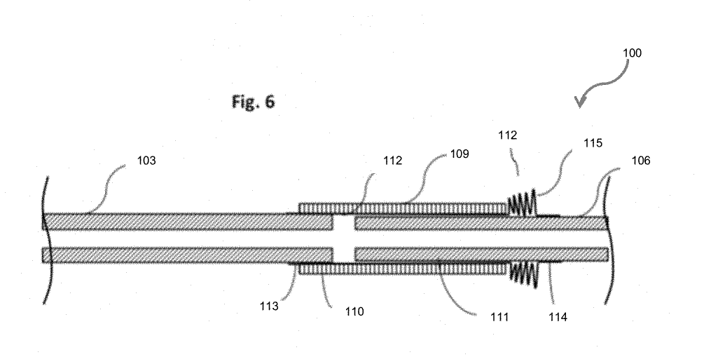

Further embodiments of the drainage catheter 100 include alternative coupling locations and orientations between the cover 112 and other catheter components. FIG. 3 is a cross sectional view that illustrates the cover 112 which is inverted under the outer telescoping component 109 at its distal end and coupled to the drainage catheter 100 at site 125. The outer telescoping component 109 is coupled to the cover 112 and/or the distal catheter component 103 at site 110. FIG. 4 illustrates a cover 112 that is coupled to the outer telescoping element 109 at site 126. FIG. 5 illustrates an embodiment similar to FIG. 4, but the cover 112 is shown drawn into the inner diameter 120 of the outer telescoping component 109. FIG. 6 shows a similar embodiment of the drainage catheter 100 shown in FIG. 2A, but the cover 112 travels through the inside diameter of the outer telescoping component 109 rather than outside of the inner diameter.

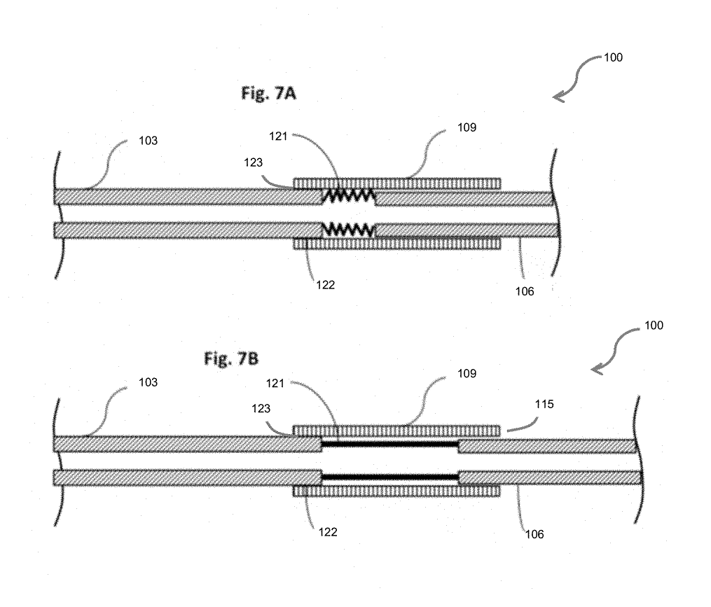

FIG. 7A is another embodiment of a drainage catheter 100 shown in the non-extended state with a flexible catheter component 122 extending from the distal catheter component 103 to the proximal catheter component 106. The flexible catheter component 122 can be coupled to the proximal catheter component 106 and distal catheter component 103 or can be a section of the drainage catheter 100 with different properties that allow it to compress and extend with longitudinal force. In this embodiment, the outer telescoping component 109 is coupled to the distal catheter component 103 at site 123. The proximal catheter component 106 is not coupled to the outer telescoping element 109. FIG. 7B illustrates that the drainage catheter 100 of FIG. 7A in the extended state, with the excess material of cover portion 115 placed under tension.

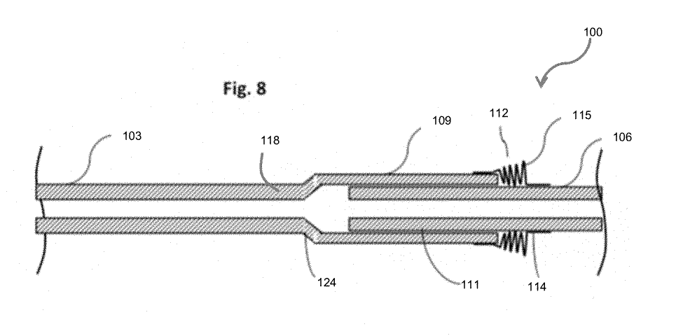

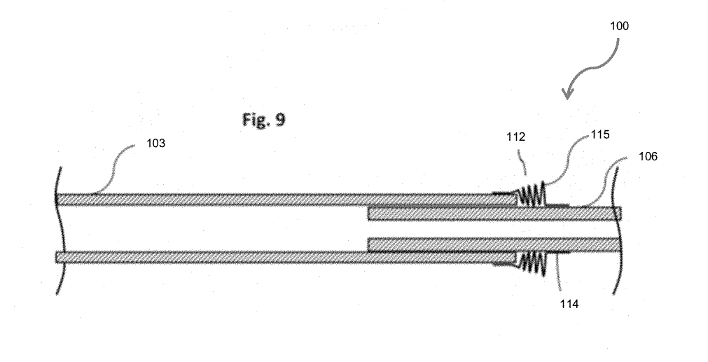

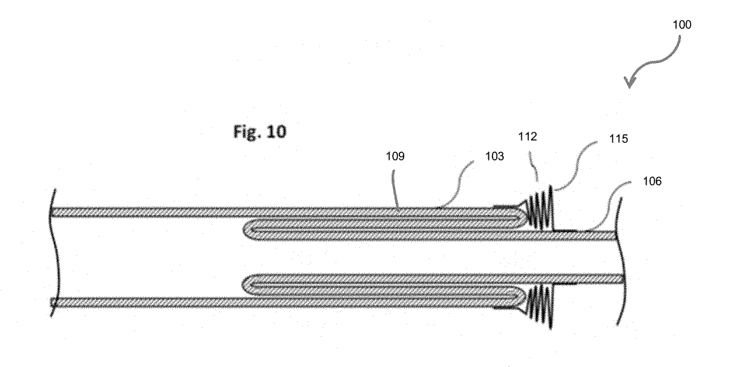

FIG. 8 illustrates another embodiment of the drainage catheter 100 where an outer telescoping component 109 is a formed portion of the distal catheter component 103. In some embodiments, a flare 124 is defined near the proximal end 118 of the distal catheter component 103 and allows for the increase in diameter. In this embodiment, the proximal catheter component 106 moves longitudinally within the outer telescoping component 109. FIG. 9 shows another embodiment of the drainage catheter 100 where the proximal catheter component 106 can move longitudinally within the distal catheter component 103, which acts as the outer telescoping component 109 in this particular embodiment. FIG. 10 shows another embodiment where the proximal catheter component 106 can invert into the distal catheter component 103, which acts as the outer telescoping component 109 in this particular embodiment.

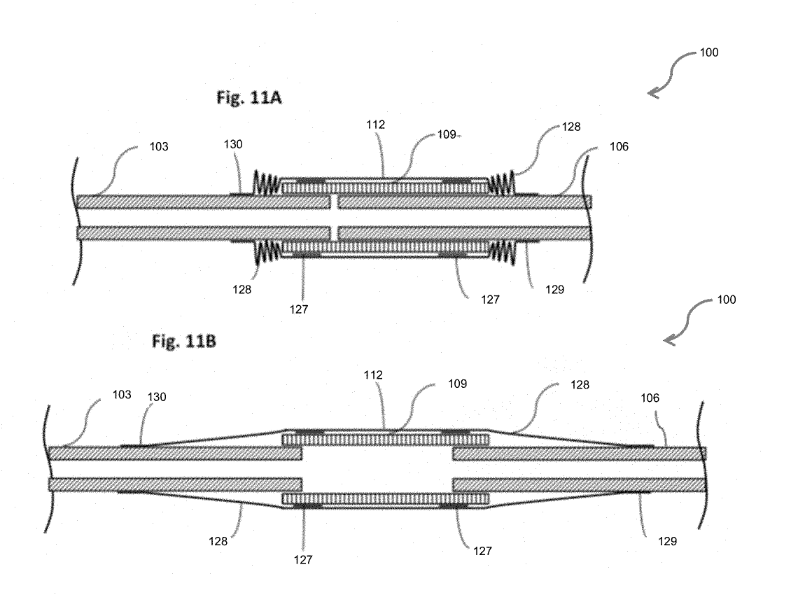

FIGS. 11A and 11B show a cross-sectional view of an additional embodiment of the drainage catheter 100 where a proximal catheter component 106 and a distal catheter component 103 both can move longitudinally within the outer telescoping component 109. In this embodiment, the cover 112 is coupled to both the proximal catheter component 106 at site 129 and to the distal catheter component 103 at site 130, while the outer telescoping component 109 is coupled to the cover 112 at site 127. In one embodiment, two separate covers are used in place of the one cover 112. The outer telescoping component 109 is not directly coupled to the proximal catheter component 106 or the distal catheter component 103. Excess cover material 128 straightens upon longitudinal extension of the drainage catheter 100 as shown in FIG. 11B.

In one aspect, minimal friction is desirable between the outer telescoping component 109 and the distal end 111 of the proximal catheter component 106. When external pressure is applied to the outer telescoping component 109 it may compress and apply a normal force to proximal catheter component 106, which may increase the static and kinetic friction. In some embodiments, the outer telescoping component 109 may include a metal tube, a coil reinforced tubing, or a braid reinforced tubing. These materials can have a sufficiently high radial strength to minimize compression and reduce the friction between the outer telescoping component 109 and the proximal catheter component 106. Additionally, in some embodiments, a material with a low coefficient of friction may be chosen for the outer telescoping component 109 and/or the distal end 111 of the proximal catheter component 106. In some embodiments, a liner with a low coefficient of friction is applied to the inner diameter of the outer telescoping component 109 and/or the outer diameter of the distal end of the proximal catheter component 106. In some embodiments, a lubricious coating or material is applied to the inner diameter of outer telescoping component 109 and/or the outer diameter of the proximal catheter component 106 in order to reduce the friction between the outer telescoping and proximal catheter components 106 and 109. Materials with low coefficients of friction that may be used include but are not limited to PTFE, ePTFE, FEP, and LDPE. Lubricious coatings and materials that may be used include but are not limited to hydrophilic coatings, PTFE, and oils.

Certain embodiments of the drainage catheter 100 may include a cover 112 with properties that facilitate its intended purpose. For example, a cover 112 can have a low flexural modulus that corresponds with high flexibility, which enables the cover 112 to compress and extend with minimal force. In some embodiments, the cover 112 can be made of various materials, including but not limited to LDPE, LLDPE, FEP, ePTFE, PTFE, PVC, and polyamide. One embodiment of the cover 112 includes wrapping a 6 mm diameter mandrel with five layers of 0.5 mil LDPE, heating to 250 degrees F. for 15 minutes, letting it cool, removing the LDPE tube from the mandrel, and stretching the tube longitudinally to draw down its diameter and decrease its flexible modulus. This results in a LDPE tube with a low flexural modulus and high longitudinal tensile strength. In one embodiment, a cover 112 is coupled to the catheter components using cyanoacrylate glue.

Preventing Catheter Component Separation

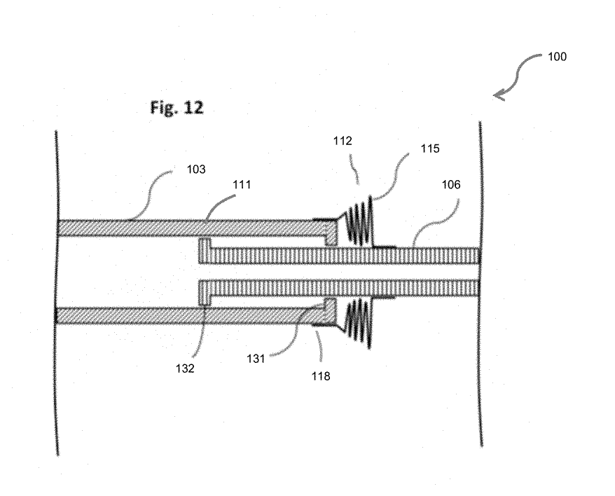

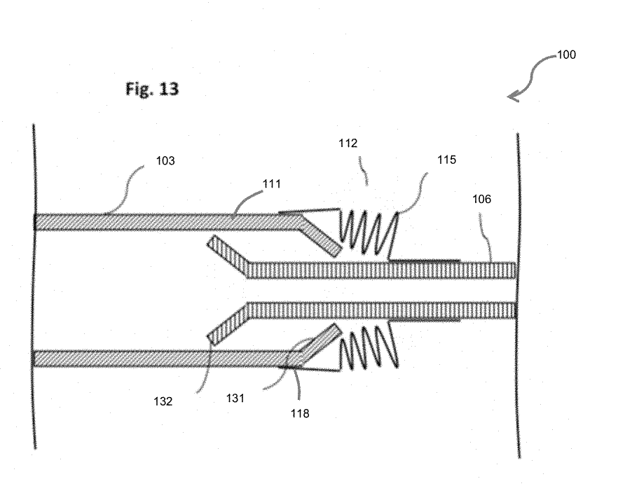

In some embodiments, the cover 112 can be manufactured with sufficient tensile strength and coupled to the appropriate catheter components to prevent component separation and minimize relative rotation of the components. In certain embodiments, additional mechanisms can be implemented as well to achieve this end. FIGS. 12 and 13 illustrate cross-sectional views of several embodiments of a telescoping element for a drainage catheter 100 where a distal catheter component 103 has a hard stop 131 formed on the proximal end 118 thereof that extends into the inner diameter. In some embodiments, the proximal catheter component 106 has a hard stop 132 formed at the distal end 111 thereof that extends out from its outer diameter. When the drainage catheter 100 is extended, the hard stop 131 of the distal catheter component 103 comes into contact with the hard stop 132 of the proximal catheter component 106 such that the catheter component separation is prevented. The hard stops 131 and 132 can be manufactured at various angles to prevent component separation, including perpendicular to the catheter components as illustrated in FIG. 12 or at an angle as illustrated in FIG. 13.

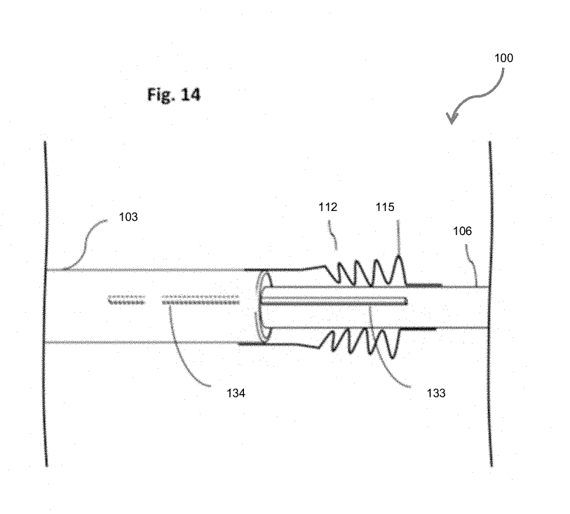

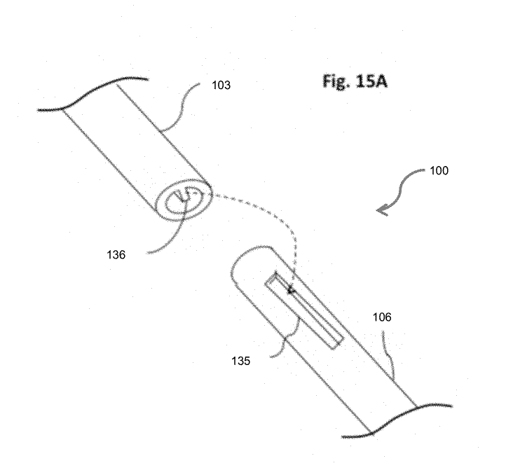

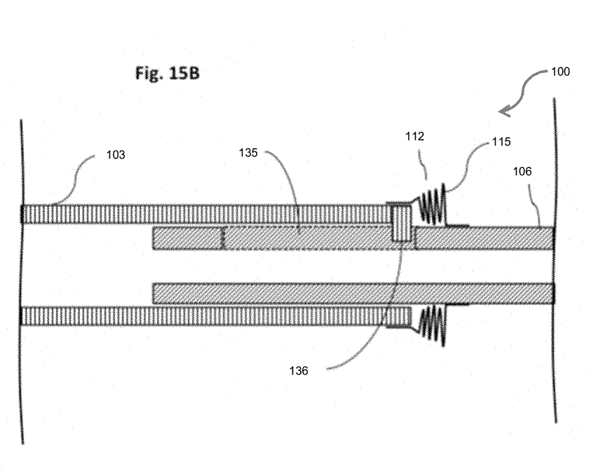

Several mechanisms to minimize rotation of the catheter components relative to each other are illustrated in FIGS. 14 and 15. FIG. 14 shows a section of proximal catheter component 106 with a raised portion 133 that can slide within a guide portion 134 in the distal catheter component 103. This interaction minimizes the rotation of the catheter components relative to each other. FIG. 15A is a perspective view of an additional embodiment of the drainage catheter 100 with the components disassembled and no cover shown. In some embodiments, the proximal catheter component 106 forms a slot 135 extending at least partway through the wall thickness that engages with a section 136, which protrudes into the inner diameter of distal catheter component 103. FIG. 15B is a cross-sectional view of the assembly shown in FIG. 15A with a cover 112 in place. The mechanism illustrated in FIGS. 15A and 15B minimizes rotation between the catheter components as well as prevents separation of the catheter components.

Associated Locking Mechanisms

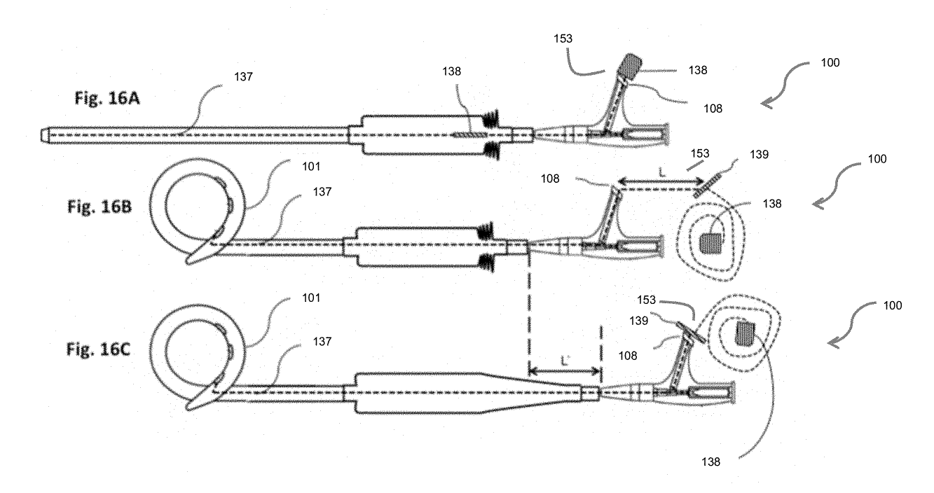

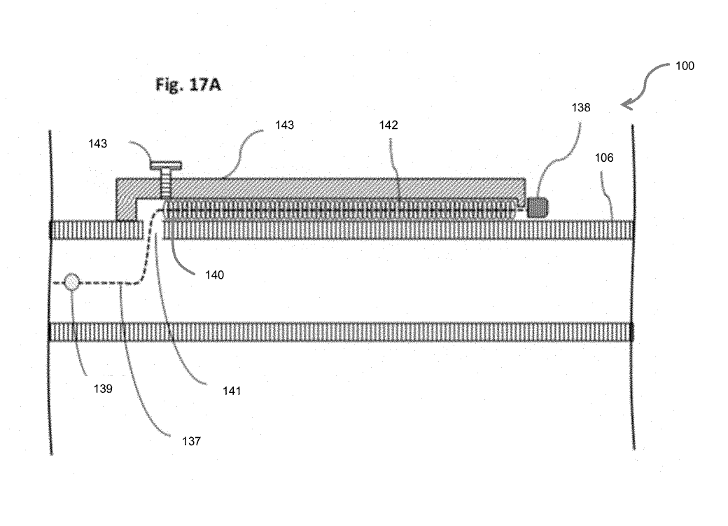

FIGS. 16A-16C show an embodiment for a locking mechanism 153 used for a telescoping catheter. As shown, the locking mechanism 153 is illustrated that places tension on the string 137 to lock the distal pigtail 101 in its shape. FIG. 16A shows a telescoping catheter introduced into the body with a string 137 extending through the interior of the catheter, coming out of the proximal hub 108 and attached to end cap 138. Lock 139 is attached to a specific longitudinal location on the string 137 but is free to rotate after being pulled past the proximal hub 108. When the cap 138 is pulled to form the pigtail loop 101 as shown in FIG. 16B, the lock 139 exits the proximal hub 108 and rotates passively, making it difficult to re-enter proximal hub 108 without manual manipulation. A length L then exists between the proximal hub 108 and the lock 139. This length L can be chosen to be near the telescoping length L′, such that when the proximal catheter component is pulled and the catheter length increases by L′ as shown in FIG. 16C, the lock 139 abuts the proximal hub 108 and places tension on string 137. The tension applied to string 137 locks the pigtail loop 101 in its looped geometry.

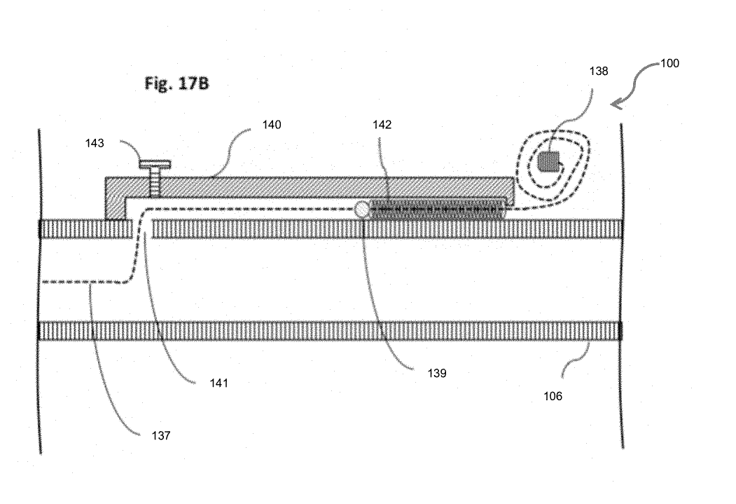

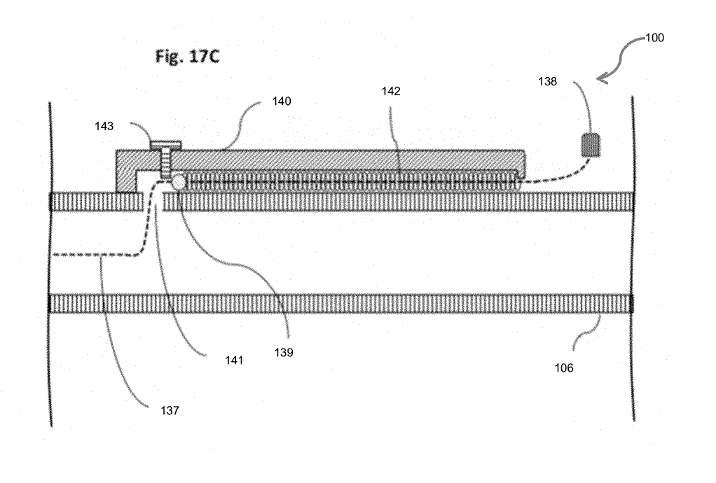

In some embodiments, an auto-locking mechanism 153 as illustrated in the cross-sectional image shown in FIG. 17A may be provided. A channel 140 is coupled to a proximal catheter component 106 and a string 137 is coupled to a lock 139. The string 137 exits the proximal catheter component 106 through an opening 141 and continues through a spring 142, which is enclosed within channel 140. String 137 is coupled to an end cap 138 at its proximal end. A locking piece 143 is coupled to channel 140 without entering its lumen. As shown in FIG. 17B, when the end cap 138 is pulled, the string 137 and lock 139 are pulled into the channel 140. Pulling the string 137 forms a shape on the distal end 117 of the drainage catheter 100. The lock 139 compresses the spring 142 as the end cap 138 is pulled. FIG. 17C shows the drainage catheter after locking piece 143 is pushed into the lumen of channel 140 and the end cap 138 is released. The spring 142 pushes against the lock 139 until the lock 139 engages with the locking piece 143. This engagement creates a specified amount of slack in string 137 that does not restrict lengthening of the drainage catheter 100.

FIGS. 16, 17A, 17B, and 17C illustrate various embodiments of auto-locking mechanisms. To those skilled in the art, it will be evident that there are many additional mechanisms to lock the string in place at a desired location, and even at a location such that the length L does not have to be near the length L′ of the drainage catheter 100. Additional embodiments include proximal catheter component 106 or hub components that couple to the string 137 or otherwise lock the string 137 in place at a desired location such that tension is placed on the string when the catheter is extended, which locks the distal catheter component 103 geometry. The string 137 can be comprised of variety of materials including, but not limited to fibers, films, plastics, and metals.

### Function

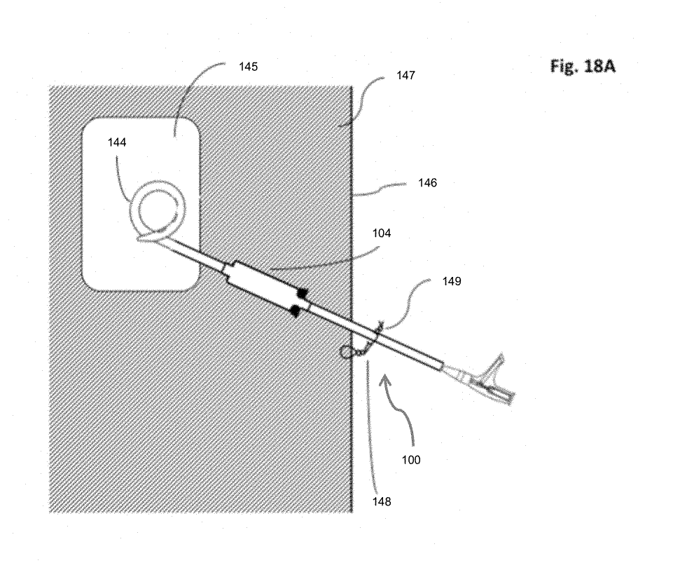

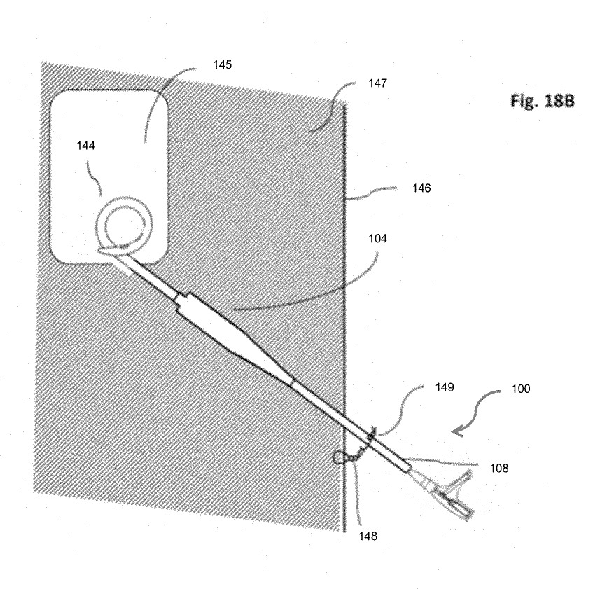

FIGS. 18A and 18B illustrate the function of a drainage catheter. Upon implantation, the distal end 144 of a drainage catheter 100 is in a body cavity 145. The body cavity 145 is beneath the surface of the skin 146 and in the body tissue 147. The drainage catheter 100 is attached to the skin via a catheter suture 149 and a skin suture 148. The telescoping element 104 of the telescoping drainage catheter 100 is in the non-extended state shown in FIG. 18A. In FIG. 18B, the telescoping drainage catheter 100 is shown in the extended stated. The distance between the body cavity 145 and the skin suture 148 has increased, for example through motion of the skin 146, motion of the body tissue 147, motion of the body cavity 145, or longitudinal tension placed on the telescoping drainage catheter 100. The telescoping element 104 has become extended to accommodate the extra length instead of undergoing catheter dislodgement or withdrawal.

### Conclusions and Ramifications

Thus, at least one embodiment of the telescoping drainage catheter provides a more reliable and economical method that can help reduce catheter dislodgement and/or withdrawal.

While the above description contains much specificity, this should not be construed as limitations on the scope, but rather as an exemplification of several embodiments thereof. Many other variations are possible. For example, the telescoping element may be reversed such that the outer telescoping component 109is attached to the proximal catheter component 106 rather than the distal catheter component 103. Also, a variety of materials, diameters, shapes, lengths, and coupling mechanisms can be used for the outer telescoping component 109. The overall length of the drainage catheter 100 can vary depending on the intended end use, and multiple diameters including but not limited to 2 Fr-20 Fr can be designed with the various embodiments of the invention. The locking mechanism 153 can have variety of shapes and orientations, including circumferentially around the catheter or at an angle along the catheter. Various locks and locking piece shapes and designs can be used, including one-way valves that act as a locking piece. Combinations of components and elements may be used, including distal catheter shapes (for example, pigtail, Malecot, Pezzer), telescoping elements, and locking mechanisms or lack thereof.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A catheter comprising:
a catheter body defining a distal catheter component defining a distal end and a proximal end in communication with a first lumen and a proximal catheter component defining a distal end and a proximal end in communication with a second lumen;
a telescoping element comprising an outer telescoping component engaged between the proximal end of the distal catheter component and the distal end of the proximal catheter component; and
a cover coupled to the distal catheter component, wherein a portion of the cover defines an excess cover length portion configurable between an extended state and a non-extended state, wherein the distal end of the proximal catheter component is slidably disposed within the outer telescoping component.

2. The catheter of claim 1, wherein the excess cover length portion of the outer telescoping component is configured to achieve a non-extended state and retract towards the telescoping element and away from the proximal catheter component such that the space between the proximal catheter component and the distal catheter component is increased.

3. The catheter of claim 1, wherein the excess cover length portion of the outer telescoping component is configured to achieve an extended state wherein the excess cover length portion is straightened, the distal edge of the proximal catheter component is confined within the outer telescoping component, and the cover has reached its maximum length.

4. The catheter of claim 1, wherein the cover extends over the outer telescoping component and is coupled to the proximal catheter component.

5. The catheter of claim 1, wherein the cover is inverted under the outer telescoping component at the distal end and coupled to the proximal catheter component.

6. The catheter of claim 1, wherein the distal end of the distal catheter component defines a pigtail loop portion having a loop-shaped configuration and wherein the pigtail loop portion defines a plurality of drain holes in communication with the first lumen of the distal catheter component and is in fluid flow communication with a lumen of the outer telescoping component.

7. The catheter of claim 1, further comprising:
a proximal hub coupled to the proximal catheter component and in fluid flow communication with the second lumen.

8. The catheter of claim 1, wherein when the distal catheter component is in the non-extended state the proximal end of the distal catheter component is in direct contact with the distal end of the proximal catheter component such that the first lumen of the distal catheter component is in fluid flow communication with the second lumen of the proximal catheter component.

9. The catheter of claim 1, wherein the distal catheter component is configured to have a hard stop formed on the proximal end thereof that extends into an inner diameter of the distal catheter component.

10. The catheter component of claim 1, wherein the proximal catheter component is configured to have a hard stop formed at the distal end thereof that extends out from an outer diameter of the proximal catheter component.

11. The catheter of claim 1, wherein the proximal catheter component and the distal catheter component are operable to move longitudinally within the outer telescoping component between the extended and non-extended positions.

12. The catheter of claim 1, wherein one or more radiopaque markers are configured to be integrated to the outer telescoping component.

13. The catheter of claim 1, further comprising a hydrophilic or hydrophobic coating along the proximal catheter component and distal catheter component.

14. The catheter of claim 1, wherein a liner with a low coefficient of friction is applied to an inner diameter of the outer telescoping component or to an outer diameter of a distal end of the proximal catheter component.

15. A method of manufacturing a catheter, the method comprising:
constructing a catheter body having a distal catheter component forming a first lumen and a proximal catheter component forming a second lumen;
engaging a telescoping element including an outer telescoping component between the distal catheter component and the proximal catheter component;
slidably disposing a distal end of the proximal catheter component within the outer telescoping component, and
coupling a cover to the distal catheter component, wherein a portion of the cover defines an excess cover length portion configured to be configured between an extended state and a non-extended state.

16. The method of claim 16, further comprising forming a flare near a proximal end of the distal catheter component.

17. The method of claim 16, further comprising constructing the cover by wrapping a 6 mm diameter mandrel with five layers of 0.5 mil LPDE, heating to 250 degrees F. for 15 minutes, letting the mandrel and LPDE configuration cool, removing the LPDE tubing from the mandrel, and stretching the tubing longitudinally.

18. The method of claim 16, further comprising applying a liner with a low coefficient of friction to an inner diameter of the outer telescoping component or to an outer diameter of a distal end of the proximal catheter component.

19. The method of claim 16, wherein the outer telescoping component comprises a coil reinforced tubing or braid reinforced tubing.

20. A locking mechanism comprising:
a telescoping catheter comprising a string extending through an interior body of the telescoping catheter and exiting out of a proximal hub and attached to an end cap;
a mechanism configured to place tension on the string and lock a distal pigtail loop; and
a lock configured to a specific longitudinal location on the string and further configured to exit the proximal hub and to rotate passively when the end cap is pulled to form the distal pigtail loop.
